(19) United States
(12) Patent Application Publication
(10) Pub. No.: US 2008/0033307 A1
(43) Pub. Date: Feb. 7, 2008

(54) INTERMITTENT PNEUMATIC COMPRESSION DEVICE WITH NON-INVASIVE BLOOD PRESSURE MONITORING

(76) Inventors: Jody A. Baudoin, Tampa, FL (US); Bruce A. Friedman, Tampa, FL (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/459,509

(22) Filed: Jul. 24, 2006

(51) Int. Cl. A61B 5/02

(52) U.S. Cl. 600/490; 600/492

(57) ABSTRACT

A limb compression system for increasing the flow of venous blood in a patient comprises an inflatable cuff, a pump, a controller, and a pressure transducer. The system collects a signal from the pressure transducer indicative of the pressure in the cuff and uses the signal to determine the blood pressure of the patient. A method for providing patient cardiovascular support and monitoring of patient blood pressure is also herein disclosed.

### FIELD OF THE INVENTION

The present invention is related to the field of intermittent pneumatic compression (IPC) therapy. More specifically, the present invention relates to a system that provides automated non-invasive blood pressure (NIBP) monitoring support in addition to IPC therapy.

### BACKGROUND OF THE INVENTION

The application of external compression to the limbs of a patient has been used for many years to improve cardiovascular function. Researchers and clinicians have used intermittent pneumatic compression (IPC) on a patient's extremity to augment venous blood flow, which helps to prevent deep vein thrombosis (DVT) and pulmonary embolism (PE), two life threatening cardiovascular complications.

IPC is a mechanical method of delivering compression to the limbs. One or more pneumatic sleeves are attached to an extremity of a patient's body. A source of pressurized gas is connected to the pneumatic sleeve and the sleeve is inflated and deflated in a cyclic fashion. This wave-like motion helps to increase the venous blood flow, thus returning the blood to the patient's heart and reducing pooling of blood in the patient's extremities. Besides the mechanical effects of enhancing venous blood flow, IPC devices have also been found to cause an increase in the body's own anti-coagulant secretions, further improving blood circulation. Deep vein thrombosis (DVT) is caused by blood clotting in a vein of the inner thigh or leg. Typically, DVT occurs when a person spends long stretches immobilized or in cramped conditions. Therefore, patients recovering from major surgery and/or leg-related surgery, such as a hip replacement, are at increased risk for DVT due to being confined in a hospital bed. Blood clots can break off from the main clot in the leg and make their way up the blood stream to the lung, resulting in a pulmonary embolism (PE) that has the potential of causing respiratory distress and failure. These conditions are exacerbated in situations where, due to excessive post-surgery bleeding, a patient is not able receive anti-coagulant drugs such as COUMADIN® to treat the DVT.

Automated blood pressure monitoring has rapidly become an accepted and, in many cases, essential aspect of patient treatment. Such monitors are now a conventional part of the patient environment in emergency rooms, intensive and critical care units and in the operating theater.

During the use of a conventional non-invasive blood pressure (NIBP) monitoring system, the blood pressure cuff is placed around the arm of a patient and is inflated to a supra-systolic pressure to fully occlude the brachial artery. The cuff is then progressively deflated and a pressure transducer in the cuff detects the pressure pulses as blood begins to flow past the pressure cuff. The data from the pressure sensor is used to compute the patient's systolic pressure, mean arterial pressure (MAP) and diastolic pressure. Furthermore, if blood pressures are monitored at multiple patient extremities, these blood pressures may be compared in a useful determination of arterial function.

In a typical hospital setting, especially that of a patient recovering from a major surgery, the patient is surrounded by a multitude of monitoring, diagnostic, and therapeutic devices. Each of these devices is connected to the patient at one or more sites, creating a vast network of patient connections surrounding the patient. This decreases patient mobility if the patient needs to be moved to a different location within the facility and also impedes clinician access to a patient to provide care. Furthermore, due to facility resources, some machines, such as a NIBP monitoring device, do not exist in a one-to-one relationship with the patients and therefore must be moved about the facility and reconnected to each new patient to perform the diagnostic measurement. Therefore, it is desirable in the field to provide a therapeutic IPC device with an integrated NIBP monitor such that the number of connections to the patient is reduced and the number of required bedside devices is also reduced.

### SUMMARY OF THE INVENTION

The following describes an apparatus for providing intermittent pneumatic compression (IPC) that further comprises an integrated non-invasive blood pressure (NIBP) monitoring device. In a first embodiment, an inflatable cuff is removably secured to an extremity of a patient that requires IPC therapy. During the IPC therapy, a source of pressurized air will selectively inflate the inflatable cuff to a first target pressure. Then the cuff is deflated until the pressure in the cuff reaches a second, lower target pressure. The IPC therapy will continue in this cyclic fashion. A pressure transducer disposed in fluid communication with the cuff continuously monitors the pressure inside the cuff for oscillometric pulses during each deflation of the cuff. The patient's blood pressure is calculated from the characteristics of these pulses.

In another embodiment of the present invention, the apparatus of the present invention operates in two modes. In a first mode, the inflatable cuff cyclically inflates to a target pressure and deflates from the target pressure to provide IPC therapy to the patient. In the second mode, in a single cycle the apparatus of the present invention inflates the cuff to a second target pressure suitable for the determination of the patient's blood pressure and releases the pressure within the cuff in a suitable fashion for the determination of the patient's blood pressure. While the pressure is being released from the inflatable cuff, the pressure transducer detects the oscillometric pulses within the cuff and the apparatus of the present invention determines the patient's blood pressure.

### BRIEF DESCRIPTION OF THE DRAWING

The drawings illustrate the best mode presently contemplated of carrying out the invention. In the drawings:

FIG. 1 depicts an embodiment of the present invention;

FIG. 2 depicts another embodiment of the present invention;

FIG. 3 depicts a schematic diagram of the operation of an embodiment of the present invention;

FIG. 4 depicts a schematic diagram of the operation of an embodiment of the present invention.

### DETAILED DESCRIPTION

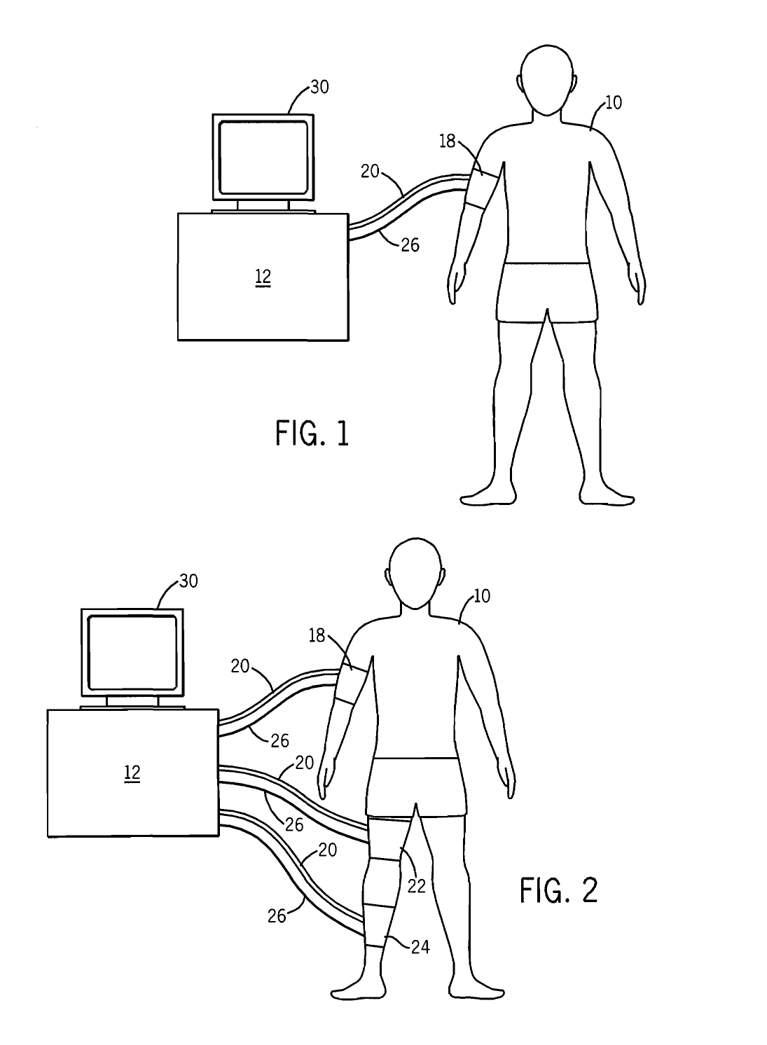

FIG. 1 generally depicts a patient connected to an embodiment of the system of the present invention. The system comprises an apparatus 12 which may comprise, as depicted in FIG. 3, a CPU 14 and an air pump 16. The air pump 16 is connected to the patient interface, which in a preferred embodiment is an inflatable cuff 18, via an air hose 20. The inflatable cuff 18 may be placed on the upper arm, as depicted in FIG. 1, or, as depicted in FIG. 2, may be a thigh cuff 22 or a calf cuff 24.

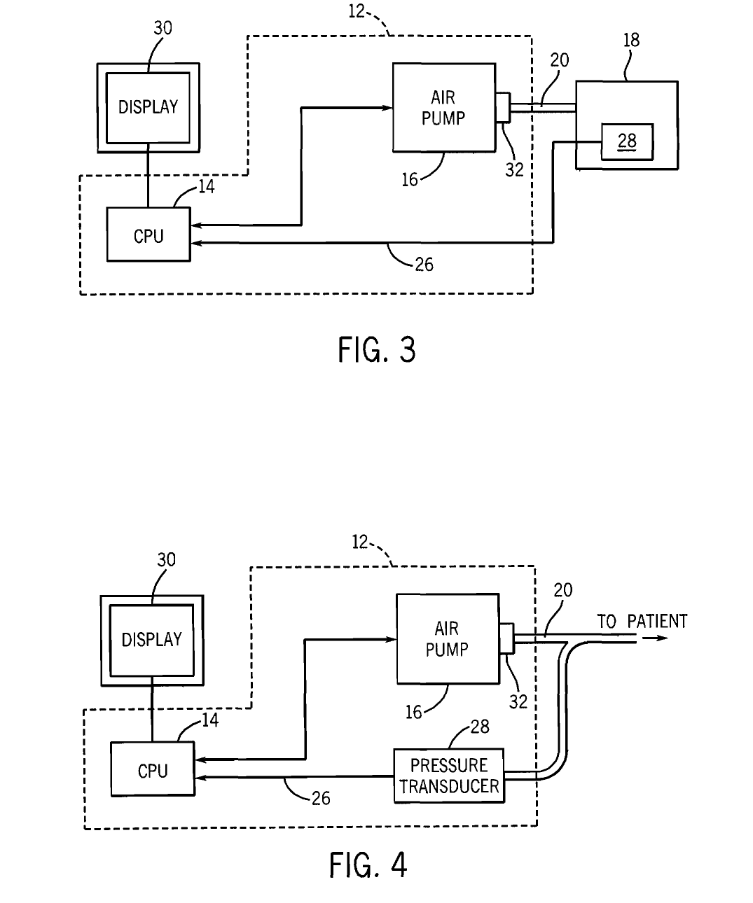

A pressure transducer (not pictured) is associated with each cuff 18 used in the system. The pressure transducer may be disposed inside the cuff 18 and a lead 26 connects the pressure transducer to the CPU 14 of the apparatus 12, as depicted in FIG. 3. It is understood that this connection may be done in a variety of ways including using wireless technology such as WIFI, RF, and infrared communication systems. Alternatively, as depicted in FIG. 4, the pressure transducer 28 may be a part integral with the apparatus 12 disposed in such fluid communication with the air hose 20 to measure the pressure within the cuff 18.

The apparatus 12 is further connected to a display 30 which may comprise a user interface such that measured patient data can be displayed to a clinician, and a clinician may modify the treatment and/or monitoring provided to the patient 10 by the apparatus 12.

The operation of the embodiment of the system depicted in FIGS. 1 and 3 is described herein. The CPU 14 in the apparatus 12 directs the air pump 16 to deliver a target pressure of air via the air hose 20 to the cuff 18. This air pressure inflates the cuff 18 to the target pressure. When the target pressure within the cuff 18 has been achieved, a pressure release valve 32 releases the pressure in cuff 18. This process is performed in a cyclical fashion as controlled by the CPU 14 to provide IPC to the patient. As in accordance with an embodiment of the present invention, when the cuff 18 has reached the target pressure, the pressure transducer (not pictured) begins to monitor the pressure in the cuff 18 and send this signal via lead 26 back to the CPU. Typically, the IPC cycle lasts between 10 and 20 seconds with the target pressure ranging between 40 and 140 mmHg. The pressure signal received by the CPU contains minute fluctuations based upon the flow of the patient's blood through the arm as it is enclosed in the cuff 18. By analyzing these fluctuations, the CPU 14 is able to determine the systolic and diastolic blood pressures of the patient as is well known in the art.

In an alternative embodiment of the present invention, the system 12 provides IPC support to the patient 10 as described above inflating the inflatable cuff 18 to a target pressure and then releasing the pressure within the cuff in a cyclical fashion. However, when a clinician desires to obtain a measurement of NIBP, the clinician activates the CPU 14 to record an NIBP measurement. To obtain an NIBP measurement, the CPU 14 directs the air pump 16 to inflate the cuff 18 to a second target pressure that is targeted for obtaining a quality measurement of NIBP. This pressure may be typically on the range of 120 mmHg to 200 mmHg. Upon achieving the second target pressure, the pressure release valve 32 releases the pressure in the cuff in a manner suitable for the detection of NIBP. This pressure release may be continuous deflation while the pressure transducer (not pictured) detects for the oscillations in the pressure inside the cuff 18. Alternatively, the pressure release valve 32 may release the pressure in the cuff 18 in a series of incremental steps while waiting to detect the pressure oscillations at each step, thus facilitating the detection of the patient's blood pressure. The calculation of the blood pressure can be determined in accordance with the teachings of Medero et al in U.S. Pat. No. 4,543,962, of Medero in U.S. Pat. No. 4,546,775, of Hood, Jr. et al in U.S. Pat. No. 4,461,266, of Ramsey, III et al in U.S. Pat. No. 4,638,810, of Ramsey III et al in U.S. Pat. No. 4,754,761, of Ramsey III et al in U.S. Pat. No. 5,170,795, of Ramsey III et al in U.S. Pat. No. 5,052,397, of Medero in U.S. Pat. No. 5,577,508 and of Hersh et al in U.S. Pat. No. 5,590,662, all of which are commonly assigned herewith and the disclosures of which are hereby incorporated by reference. In any event, it is desirable to use any of the known techniques to determine the quality of the oscillation complexes received at each cuff pressure so that the blood pressure determination is made using the physiological relevant cuff pressure oscillations from each heart beat and not artifacts.

In a still further embodiment of the present invention, as depicted in FIG. 2, multiple inflatable cuffs may be applied to the patient 10. These cuffs may be an arm cuff 18, a thigh cuff 22, a calf cuff 24, or an ankle cuff (not pictured). An embodiment of the present invention utilizes the thigh cuff 22 and the calf cuff 24, in a typical arrangement for the application of IPC to a patient's leg. It is contemplated within the present invention that a thigh cuff 22 and calf cuff 24 may be also applied to the patient's other leg at the same time. In an arrangement comprising a thigh cuff 22 and a calf cuff 24, the system of the present invention can monitor the blood pressure at two points in a single patient extremity. These two blood pressure measurements can be compared to determine blood pressure difference within the single extremity. This indication may provide useful information to a clinician in monitoring the progression of an IPC treatment.

Alternatively, arm cuff 18 may be used in conjunction with calf cuff 24. It is also contemplated within this embodiment that arm cuff 18 may be utilized with thigh cuff 22, thigh cuff 22 and calf cuff 24, or the ankle cuff. In this embodiment of the present invention, the blood pressure in both the arm and the leg are collected by the CPU 14. The comparison of these pressures detected from two different extremities of the patient 10 provides an indication of patient arterial function and cardiac health that is useful to the clinician in monitoring the treatment and progression of the patient 10. One such indication is the ankle-brachial index wherein a determination of the presence of blood clots in the patient's leg is made by comparing the patient's blood pressure at the arm versus at the ankle while the patient is lying down. A lower blood pressure in the ankle than the arm is indicative of blood vessel blockage.

The presently disclosed invention provides the advantage that it combines a system for providing a commonly used patient therapy, with a system for providing a commonly used patient monitoring process. This reduces the number of machines that must be in place in a single room and furthermore reduces the total number of patient connections that are required to provide treatment and monitoring to a patient in recovery from surgery. Furthermore, the advantage of collecting blood pressure measurements at multiple extremities provides the clinician with a useful diagnostic tool that under current systems was not available. Therefore, the present embodiment increases the quality of information provided to a clinician to aid in the clinician's monitoring and treatment decisions surrounding the patient.

This written description uses examples to disclose the invention, including the best mode, and also to enable any person skilled in the art to make and use the invention. The patentable scope of the invention is defined by the claims and may include other examples that occur to those skilled in the art. Such other examples are intended to be within the scope of the claims if they have structural elements that do not differ from the literal language of the claims, or if they include equivalent structural elements of insubstantial difference from the literal language of the claims.

Various alternatives and embodiments are contemplated as being with in the scope of the following claims, particularly pointing out and distinctly claiming the subject matter regarded as the invention.

What is claimed is:

1. A therapeutic circulatory support device for increasing the flow of venous blood in a patient, the device comprising:
a limb compression system comprising:
an inflatable cuff formed for attachment to a limb or other extremity of the patient;
a pressurized air source for providing pressure to the inflatable cuff;
a controller for controlling the amount of pressure in the inflatable cuff; and
a pressure transducer disposed in fluid communication with the cuff for detecting pressure oscillations within the cuff;
wherein the controller operates in a first mode where the pressure in the cuff is maintained in a cyclical pattern and a second mode in which the controller determines the patient's blood pressure based upon the detected pressure oscillations.

2. The device of claim 1 wherein when the controller is in the first mode, the controller inflates the cuff to a first target pressure and deflates the cuff in a cyclic manner to perform intermittent pneumatic compression on the limb.

3. The device of claim 2 wherein when the controller is in the second mode, the controller inflates the cuff to a second target pressure and deflates the cuff to determine the patient's blood pressure.

4. The device of claim 3 wherein the controller deflates the cuff in the pattern of a step function to determine the patient's blood pressure.

5. A limb compression system for increasing the flow of venous blood in a patient, the system comprising:
at least one inflatable cuff formed for attachment to the patient;
a pump for providing pressure to the inflatable cuff;
a controller for controlling the amount of pressure in the inflatable cuff; and
a pressure transducer disposed in fluid communication with the cuff for detecting pressure oscillations within the cuff,
wherein the controller collects a signal from the pressure transducer indicative of the pressure oscillations in the cuff and uses the signal to determine the blood pressure of the patient.

6. The system of claim 5 wherein the system includes two cuffs attached to two patient extremities such that the controller collects signals from each pressure transducer to determine blood pressure measurements at both cuffs.

7. The system of claim 6 wherein the controller compares the two measurements of blood pressure to determine an indication of patient health.

8. The system of claim 5 wherein the controller inflates the cuff to a first target pressure, the first target pressure being defined by a clinician and then deflates the cuff to provide circulatory support for the patient.

9. The system of claim 8 wherein the controller inflates and deflates the cuff in a regular cyclical pattern.

10. The system of claim 9 wherein the controller continuously monitors the patient blood pressure.

11. The system of claim 9 wherein the controller intermittently monitors the patient blood pressure.

12. The system of claim 11 wherein the controller inflates the cuff to a second target pressure when patient blood pressure is to be determined.

13. The system of claim 12 wherein the controller modifies a cuff deflation rate when determining patient blood pressure.

14-20. (canceled)
